(19) United States
(12) Patent Application Publication
(10) Pub. No.: US 2024/0285935 A1
(43) Pub. Date: Aug. 29, 2024

(54) CARDIAC ASSIST SYSTEM WITH FLOW GUIDING NOZZLE

(71) Applicant: KARDION GMBH, Stuttgart (DE)

(72) Inventors: Vladimir Popov, Stuttgart (DE); Marvin Mitze, Stuttgart (DE)

(21) Appl. No.: 18/572,481

(22) PCT Filed: Jun. 29, 2022
(86) PCT No.: PCT/US2022/035550

§ 371 Date: Dec. 20, 2023

Related U.S. Application Data
(60) Provisional application No. 63/202,996, filed on Jul. 2, 2021.

(51) Int. Cl. A61M 60/90; A61M 60/17; A61M 60/216; A61M 60/411; A61M 60/81; A61M 60/857

(52) U.S. Cl. CPC A61M 60/90; A61M 60/17; A61M 60/216; A61M 60/411; A61M 60/81; A61M 60/857; A61M 2205/04; A61M 2205/103; A61M 2206/20; A61M 2210/125

(57) ABSTRACT

A cardiac assist system for pumping blood which can be introduced into a blood vessel through a catheter. The system comprises the pump, a pump housing and a tube connected to the pump housing. An inlet guide nozzle in fluid communication with the tube may have a minimum-width constriction, e.g. located at about 50%, or more or less, of the length of the nozzle in the flow direction. The nozzle may have a curved contour protruding into the flow channel with a single concavity or convexity along an entire length thereof. A distal lip of the nozzle may be curved.

### BACKGROUND

### Field

This development relates to a cardiac assist system, in particular to such a system having a flow guiding nozzle.

### Description of the Related Art

Cardiac support systems are capable of taking over the pumping function of the human heart partially or completely by moving human blood from an anterior chamber of the heart into the aorta. The efficient delivery of blood from a ventricle into the aorta by means of a cardiac assist system requires that flow losses caused by turbulence in the cardiac assist system be prevented or minimized.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for cardiac assist systems.

The following disclosure describes non-limiting examples of some embodiments. For instance, other embodiments of the disclosed systems and methods may or may not include the features described herein. Moreover, disclosed advantages and benefits can apply only to certain embodiments and should not be used to limit the disclosure.

Described herein are features for a cardiac support system that ensures the efficient continuous delivery of blood from a ventricle to the aorta. Various embodiments are described for a cardiac assist system for pumping blood which can be introduced into a blood vessel through a catheter. The system comprises the pump, a pump housing and a tube connected to the pump housing. An inlet guide nozzle in fluid communication with the tube may have a minimum-width constriction located at 50% or more of the length of the nozzle in the flow direction. The constriction may be located at about 50%, less than 50%, or more than 50% of the length of the nozzle. The nozzle may have a curved contour protruding into the flow channel with a single concavity or convexity along an entire length thereof. A distal lip of the nozzle may be curved.

In one aspect, a cardiac support system is described. The system comprises a housing, a hollow body, a distal end portion, an inlet portion, and a guide nozzle. The housing comprises a pump configured to be introduced into a blood vessel through a catheter to pump blood from the left ventricle into the aorta. The hollow body is connected to the housing and comprises a flexible hose portion with a delivery channel extending therethrough to the pump. The distal end portion is connected to the hollow body, and the inlet portion is located between the distal end portion and the hollow body. The inlet portion has at least one inlet opening configured to receive blood therethrough. The guide nozzle extends from a nozzle inlet opening, that is in fluid communication with the at least one inlet opening of the inlet portion, to an outlet opening that is in fluid communication with the delivery channel of the flexible hose portion. The guide nozzle is connected to a distal end of the hose portion and is facing the distal end portion. The guide nozzle protrudes radially-inwardly along a contour to form a minimum-width constriction located between the nozzle inlet opening and the nozzle outlet opening, and the contour has a single, continuous concavity or convexity along an entire length from the nozzle inlet opening to the nozzle outlet opening.

Various embodiments of the above or other aspects may be implemented. The guide nozzle may have a curved distal opening edge surrounding the inlet opening. The opening edge may at least partially define the at least one inlet opening. The guide nozzle may be inserted into the hose section. The guide nozzle may be formed as a one-piece, molded part. The guide nozzle may be composed of a plurality of shell-shaped moldings. The guide nozzle may define a nozzle channel extending along a nozzle axis having cross-sectional areas as measured perpendicular to the nozzle axis, and where the cross-sectional areas decrease from the inlet opening to the constriction and increase from the constriction to the outlet opening. The cross-sectional areas may be circular or elliptical.

Further, the guide nozzle may have a flow guide contour, located in the plane of a longitudinal section running along the nozzle axis in a Cartesian coordinate system with a coordinate origin lying on the nozzle axis and an abscissa lying on the nozzle axis as a line drawn in a first and/or second quadrant of the Cartesian coordinate system and is formed as a convex line and as a concave line extending in a third and/or fourth quadrant of the Cartesian coordinate system. The flow guide contour may have a rounded apex facing the distal end portion. The convex line and the concave line may each be continuously differentiable. The cardiac assist system may further comprise a flow guide body connected to the distal end portion and projecting proximally into the inlet portion. The flow guide body may have a guide contour which is rotationally symmetrical to the nozzle axis. The flow guide body may have a flow guiding contour, located in the plane of a longitudinal section extending along the nozzle axis in a Cartesian coordinate system with a coordinate origin lying on the nozzle axis and an abscissa lying on the nozzle axis as a line drawn in a first and/or second quadrant of the Cartesian coordinate system and is formed as a convex line and as a concave line extending in a third and/or fourth quadrant of the Cartesian coordinate system.

Further, a first axial distance AE from the inlet opening to the constriction may be greater than a second axial distance AA from the constriction to the outlet opening. The guide nozzle may comprise a stepped-down outer width configured to attach to an inner surface of the hollow body.

In another aspect, a cardiac support system is described. The system comprises a pump, a body, and a guide nozzle. The pump is configured to be introduced into a blood vessel through a catheter to pump blood from the left ventricle into the aorta. The body has a proximal end fluidly connected with the pump and extending longitudinally to a distal end to at least partially define a flow channel. The guide nozzle is located at the distal end of the body and is in fluid communication with the body to at least further partially define the flow channel. The guide nozzle protrudes radially-inwardly into the flow channel with a curvature having a single concavity or convexity along an entire length from an inlet to an outlet of the guide nozzle.

Various embodiments of the above or other aspects may be implemented. A first axial distance AE, measured from the inlet opening to a minimum-width constriction of the flow channel within the guide nozzle, may be greater than a second axial distance AA, measured from the minimum-width constriction to the outlet opening. The guide nozzle may comprise a stepped-down outer width configured to attach to an inner surface of the body. The guide nozzle may have a curved distal opening edge.

In another aspect, a cardiac assist device of the invention has a pumping device for pumping blood into a blood vessel through a catheter and contains a pump housing and a hollow body connected to the pump housing. The hollow body has a distal end portion and an inlet portion formed between the distal end portion and the pump housing with at least one suction inlet opening and comprises a flexible hose portion with a delivery channel extending to the pump housing. The at least one suction inlet port communicates with the delivery channel through a guide nozzle connected to an end of the hose section facing the distal end portion. The guide nozzle has a constriction formed between an inlet port and an outlet port.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawing, can be arranged, substituted, combined, and designed in a wide variety of different configurations, all of which are explicitly contemplated and make part of this disclosure.

FIG. 1 is a perspective view of an embodiment of a cardiac support system with a pumping device and having a guide nozzle.

FIG. 2 is a partial cross-section view of the cardiac support system of FIG. 1.

FIG. 3 is another partial cross-section view of the heart support system of FIG. 1.

FIG. 4 is a cross-section view of a nozzle body of the guide nozzle of FIGS. 1-3.

FIG. 5 is a perspective view of an embodiment of a guide nozzle that may be used with the cardiac support system of FIGS. 1-4.

FIG. 6 is an exploded view of another embodiment of a guide nozzle having two molded parts that may be used with the cardiac support system of FIGS. 1-4.

FIG. 7 is an exploded view of another embodiment of a guide nozzle having three molded parts that may be used with the cardiac support system of FIGS. 1-4.

FIG. 8 is a schematic showing an embodiment of flow lines through a portion of the cardiac assist system including the guide nozzle.

FIG. 9 is a schematic showing an embodiment of flow lines through a portion of a cardiac assist system without the guide nozzle for comparison.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

The efficiency for pumping blood from a ventricle into the aorta in a cardiac assist system, i.e. the pumping efficiency of the cardiac assist system, can be increased by preventing or at least minimizing the occurrence of turbulences in the blood flow generated by a pumping device for pumping blood. The systems described herein allow for sucking the blood conveyed from a heart chamber into the aorta in such a way that the fluid flow in the inlet section is at least largely laminar. The flow has a fluid flow profile in the delivery channel, the maximum of which lies in or at least near the center of the flow channel. Shear forces acting on the components of the blood are minimized by the systems described herein and thus damage to the red blood cells and the associated hemolysis is mitigated or prevented.

The guide nozzle may have a concave curved opening edge surrounding the inlet opening. In particular, the guide nozzle can be arranged in such a way that the opening edge delimits the at least one suction inlet opening. The guide nozzle can be inserted into the hose section of the hollow body. It is possible to design the guide nozzle as a one-piece molding. Alternatively, it is possible that the guide nozzle is composed of several shell-shaped molded parts.

The guide nozzle may have a nozzle channel extending along a nozzle axis with cross-sectional areas perpendicular to a nozzle axis, the area of which decreases from the inlet opening to a constriction or the narrow point and increases from the narrow point to the outlet opening. The cross-sectional areas can be circular or elliptical, for example.

The guide nozzle may have a flow guide contour which is formed in the plane of a longitudinal section extending along the nozzle axis in a Cartesian coordinate system with a coordinate origin lying on the nozzle axis and an abscissa lying on the nozzle axis as a convex line extending in a first and/or second quadrant of the Cartesian coordinate system and as a concave line extending in a third and/or fourth quadrant of the Cartesian coordinate system. The flow guiding contour may have a vertex facing the distal end section.

The contour may have a single concavity or convexity along its entire length. The contour may have a minimum width at a location that is greater than 50% of the axial length of the nozzle in the flow direction. The contour line may be constantly differentiable. For this purpose, the cardiac support system preferably contains a flow guide body connected to the distal end section and projecting into the inlet section. The flow guiding body can have a guiding contour which is rotationally symmetrical to the nozzle axis.

The flow guiding body may have a flow guiding contour which is formed in the plane of a longitudinal section extending along the nozzle axis in a Cartesian coordinate system with a coordinate origin lying on the nozzle axis and an abscissa lying on the nozzle axis as a convex line extending in a first and/or second quadrant of the Cartesian coordinate system and as a concave line extending in a third and/or fourth quadrant of the Cartesian coordinate system.

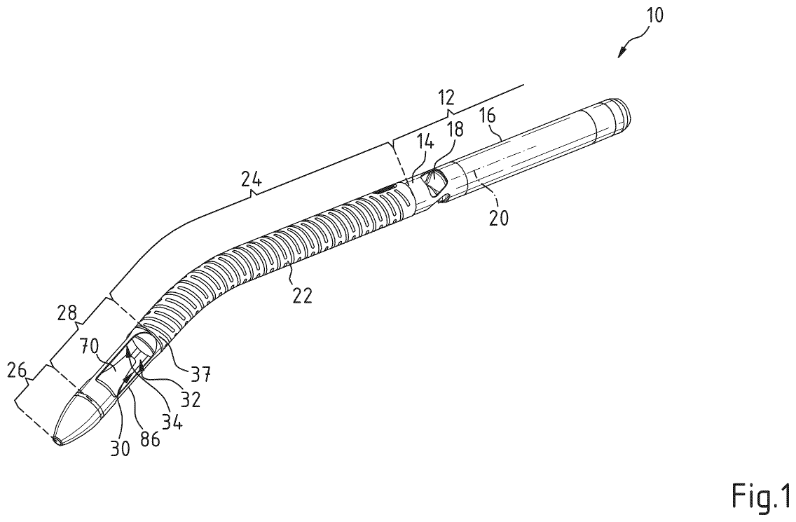

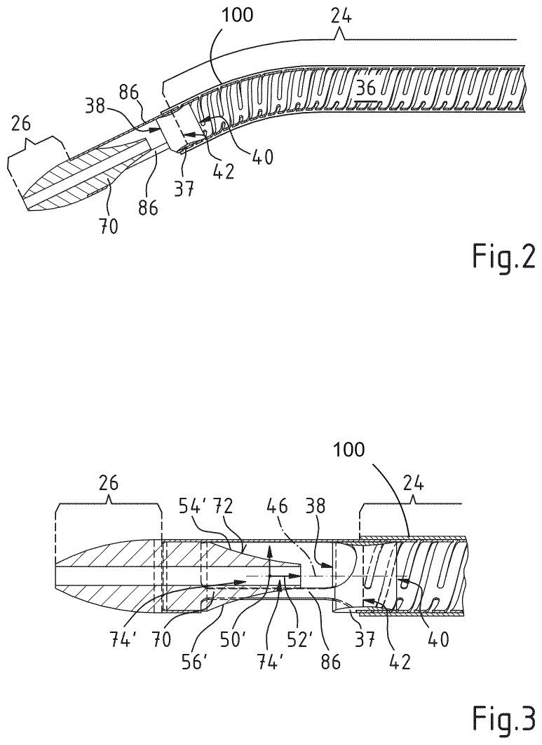

The cardiac support system 10 shown in FIG. 1 contains a pumping device 12. The system 10 can be introduced into a heart chamber through a catheter. The system 10 incudes a pump housing 14 with a delivery rotor 18 which can be driven by an electric motor 16 and has an impeller which can rotate about an axis of rotation 20. A hollow body 22 of metallic material is connected to the pump housing 14. The hollow body 22 has a flexible fluid-tight hose section 24. The section 24 may be a metallic material coated with a flexible membrane 100 made from a material such as silicone (not shown in FIG. 1 for simplicity but shown in FIGS. 2 and 3). The section 24 has an inlet section 28 located between a distal end section 26 and the flexible hose section 24.

The inlet section 28 includes a first inlet opening 30, a second inlet opening 32 and a third inlet opening 34. As shown, there are three inlet openings 30, 32, 34. There may be one, two, four, five, six, seven, eight, nine, ten, or more inlet openings. The openings 30, 32, 34 may be cutouts forming windows in the sidewall of the inlet section 28. There may be a corresponding inlet opening for each portion of the guide nozzle inlet having a curved distal edge at the nozzle opening, as further described.

FIG. 2 is a cross-section view of the cardiac support system 10. A delivery channel 36 for blood is formed in the tube section 24 of the hollow body 22 and extends to the pump housing 14. In the system 10, the inlet openings 30, 32, 34 fluidly communicate with a guide nozzle 37 connected to an end of the hose section 24 facing the distal end section 26 of the cardiac support system 10.

FIG. 3 is a partial cross-section view of the cardiac support system 10 showing the guide nozzle 37. The guide nozzle 37 has an inlet opening 38 and an outlet opening 40. The inlet opening 38 is configured to receive blood therethrough and the outlet opening is configured to allow blood to flow out of the guide nozzle 37. Between the inlet opening 38 and the outlet opening 40, the guide nozzle 37 has a constriction 42. The constriction 42 may be a portion of the nozzle at a location of minimum inner width, e.g. diameter, of the body 44. The constriction 42 may be a portion of the nozzle at a location of minimum inner width at the inlet of the device, for example within the nozzle portion. The constriction 42 may be a portion of the nozzle at a location of minimum width that is between locations maximum widths at the proximal and distal ends of the nozzle.

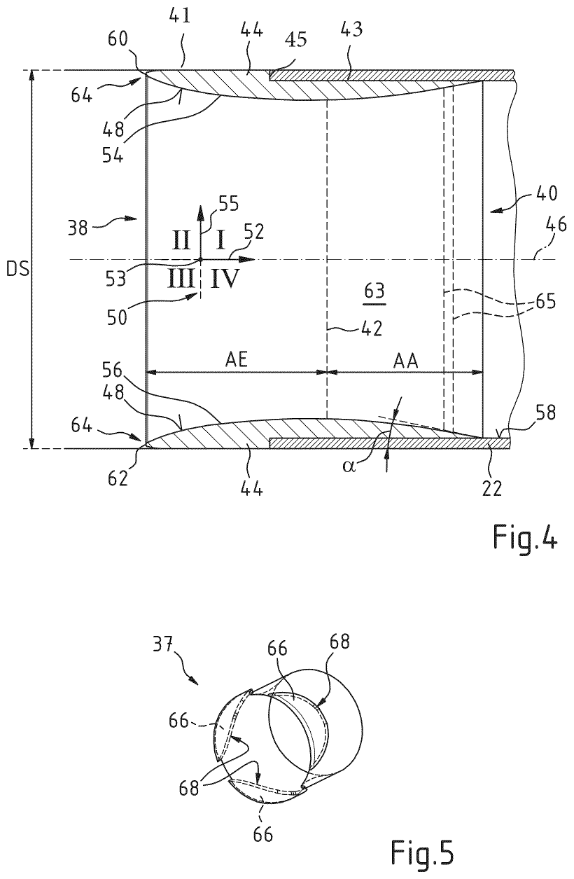

FIG. 4 shows a longitudinal cross-section of a nozzle body 44 of the guide nozzle 37 defining a nozzle axis 46. The longitudinal axial distance AE from the inlet opening 38 to the constriction 42 is greater than the longitudinal axial distance AA from the constriction 42 to the outlet opening 40. The constriction 42 may be axially located at greater than 50% of the total length of the body 44, where the total length of the body 44 is measured axially from the inlet opening 38 (e.g., the opening edge 64) to the outlet opening 40 (e.g., where a proximal end of the body 44 meets the hollow body 22). In some embodiments, the constriction 42 may be located at 50% or more, 55% or more, 60% or more, 65% or more, 70% or more, or 75% or more of the total length of the body 44 as measured axially as described from the inlet opening. In some embodiments, the constriction 42 may be locations that are greater than or less than 50% of the axial length of the body 44 from the opening. In some embodiments, the constriction 42 may be located at 52-54%, 50-55%, 48-57%, 45-60%, 40-65% or 35-70% of the axial length of the body 44 from the inlet opening. The length of the body 44 may be the length of the separate piece as shown. In embodiments where the body 44 may be integral with the hollow body 22, the axial length of the body 44 may extend from the opening 38 to the portion of the body 44 or body 22 where the inner width is constant in an axial direction.

The guide nozzle 37 is rotationally symmetrical in relation to the nozzle axis 46. It has a flow guide contour 48 on a radially-inwardly facing surface thereof. The contour 48 may be located in a plane that intersects the nozzle axis. The plane may lie within the axes of the Cartesian coordinate system 50, the x-axis or abscissa 52 of which extends along the nozzle axis 46 and the y-axis or ordinate 55 of which, starting from the coordinate origin 53, points in the radial direction relative to the nozzle axis 46. The contour 48 may be formed as a continuously differentiable convex line 54 extending in the first quadrant I and the second quadrant II of the coordinate system 50. The contour 48 may additionally be formed as a continuously differentiable concave line 56 extending in the third quadrant III and the fourth quadrant IV of the coordinate system 50. The concave line 56 may extend at an acute angle a in a proximal direction towards the channel wall 58 of the conveying channel 36 in the hose section 24 of the hollow body 22. It is understood that “concave” and “convex” are used here to describe the cross-section of the body 44 as oriented in FIG. 4. The concave line 56 and the convex line 54 may be portions of the same radially-inwardly protruding contour 48 but located at different angular locations about the axis 46. The contour 48 may have a single and continuous concavity or convexity, depending on orientation, from the inlet opening 38 to the outlet opening 40. There may therefore be no inflection points along the contour 48. The mathematical first derivative of the contour 48 in the coordinate system 50 may be positive or negative (depending on orientation) continuously from the inlet opening 38 to the constriction 42 and then negative or positive, respectively, continuously from the constriction 42 to the outlet opening 40. The mathematical second derivative of the contour 48 in the coordinate system 50 may be continuously positive or negative, depending on orientation, from the inlet opening 38 to the outlet opening 40.

The convex line 54 and the concave line 56 each have a distal apex 60, 62, which faces in a distal direction to the distal end section 26 of the cardiac support system 10. The opening edge 64 of the guide nozzle 37 surrounding the inlet opening 38 is curved, for example concavely curved. The edge 64 at least partially defines the first, second and third inlet openings 30, 32 and 34 in the inlet section 28 of the hollow body 22. The guide nozzle 37 has a nozzle channel 63 extending along the nozzle axis 46 with circular cross-sectional areas 65 perpendicular to the nozzle axis 46. The cross-sectional areas 65 of the channel 63 may decrease from the inlet opening 38 to the constriction 42. The cross-sectional areas 65 of the channel 63 may increase from the constriction 42 to the outlet opening 40. Some or all of the cross-sectional areas 65 may be circular. In some embodiments, some or all of the cross-sectional areas 65 may be elliptical.

The guide nozzle 37 includes a first outer surface 41 having a first outer width. The guide nozzle 37 includes a second outer surface having a second outer width. The second outer width is smaller than the first outer width. The second outer width is located proximally of the first outer width. The first and second outer surfaces 41, 43 are connected by a step 45. The step 45 may be perpendicular to the axis as shown. The step 45 may have a height that is the same or similar to the thickness of the sidewall of the hollow body 22. A smooth, continuous outer surface may extend along the outer surface of the hollow body 22 to the first outer surface 41. The second outer surface 43 may contact an inner surface of the hollow body 22.

The guide nozzle 37 may be a separate part that attaches to the hollow body 22. The guide nozzle 37 may be inserted into the hollow body 22. The guide nozzle 37 may be partially inserted. The guide nozzle 37 may include a stepped-down outer width, for example diameter, configured to attach to a sidewall of the hollow body 22. The step 45 may contact a distal end of the sidewall of the hollow body 22. The second outer surface 43 may contact the inner surface of the sidewall of the hollow body 22. The stepped-down feature may be part of each portion of a multi-portion guide nozzle 37. The guide nozzle 37 may be flexible such that the second inner surface 43 can flex inward to be inserted into the hollow body 22, and then flex outward to fixedly attach to the hollow body 22. Mechanical or other attachments may be used to secure the pieces together, for example adhesive.

FIG. 5 shows the guide nozzle 37 in the Cardiac Support System 10 The guide nozzle 37 is designed as a one-piece molding made of a 360° round material, which is inserted into the end of the hose section 24 of the hollow body 22 facing the distal end section 26. The molded part has a collar 66 with a shoulder 68, which forms a stop for the front end of the hose wall of the hose section 24 of the hollow body 22. In the area of the collar 66, the molded part forming the guide nozzle 37 has an external diameter corresponding to the external diameter DS of the hose section 24 of the hollow body 22.

A flow guide body 70 is connected to the distal end section 26 of the cardiac support system 10. The body 70 extends proximally with a decreasing width. The body 70 has a guide contour 72 rotationally symmetrical to the nozzle axis 46. The guiding contour 72 follows, in the plane of the longitudinal section along the nozzle axis 46 shown in FIG. 3, in a Cartesian coordinate system 74′ with a coordinate origin 50′ lying on the nozzle axis 46 and an abscissa 52′ lying on the nozzle axis 46, a convex line segment 54′ extending in the first and/or second quadrant of the Cartesian coordinate system 74′ and a concave line segment 56′ in the third and/or fourth quadrant of the Cartesian coordinate system 74′.

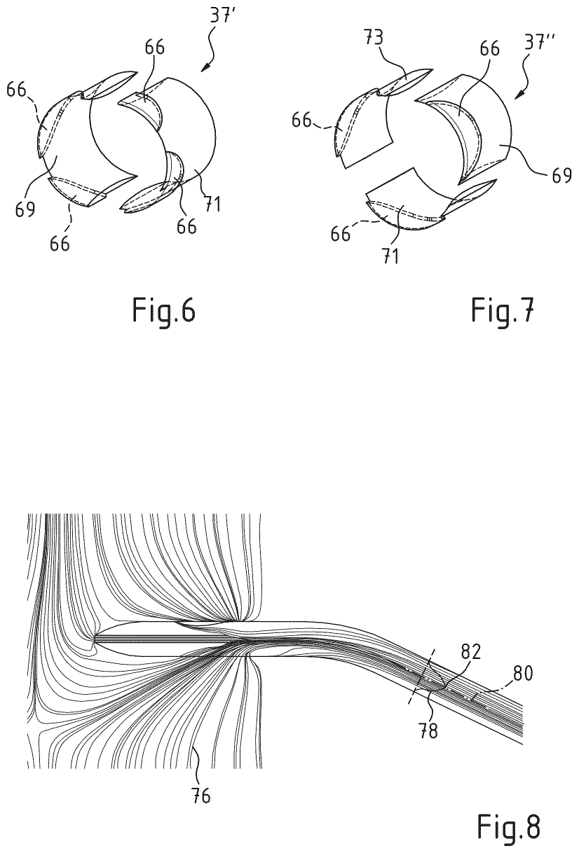

FIG. 6 shows a guide nozzle 37′ modified to form the guide nozzle 37, which is composed of a first shaped part 69 designed as a 180° shaped piece and a second shaped part 71 designed as a 180° shaped piece, which have collars 66 and can be inserted into the end of the hose section 24 of the hollow body 22 facing the distal end section 26 between the struts 86 formed therein.

FIG. 7 shows a further guide nozzle 37″ modified to form the guide nozzle 37, which is composed of a first shaped part 69 designed as a 120° shaped piece, a second shaped part 71 designed as a 120° shaped piece and a third shaped part 73 designed as a 120° shaped piece with collar 66, which can be inserted into the end of the hose section 24 of the hollow body 22 facing the distal end section 26 between struts formed therein.

FIG. 8 shows the flow lines 76 of a fluid flow through a section of the Cardiac Support System 10 having the guide nozzle 37 The arrangement and geometric shape of the guide nozzle 37 in the Cardiac Support System 10 causes the blood drawn from a heart chamber into the delivery channel 36 by the pumping device 12 to form a flow profile 78 in the delivery channel 36 over its cross-section, which has a maximum 82 located near the delivery channel center 80. The flow lines 76 of the fluid flow are caused by an optimized geometry of the guide nozzle 37 and the flow guide body 70. The concavely curved opening edge 64 of the guide nozzle 37 ensures here that the boundary layer of the conveyed blood lying against the surface of the nozzle body 44 is stabilized, since flow separations in the area of the inlet opening 38 of the guide nozzle 37 are minimized. The increasing narrowing of the nozzle channel 63 in the nozzle body 44 from the inlet opening 40 to the narrowing 42 is optimized in such a way that no breaks in fluid flow occur here either. The increase in the free cross-section of the nozzle channel 63 from the constriction 42 to the outlet opening 40 corresponds to a long outlet geometry and is thus designed in such a way that flow separations caused by pressure gradients are avoided.

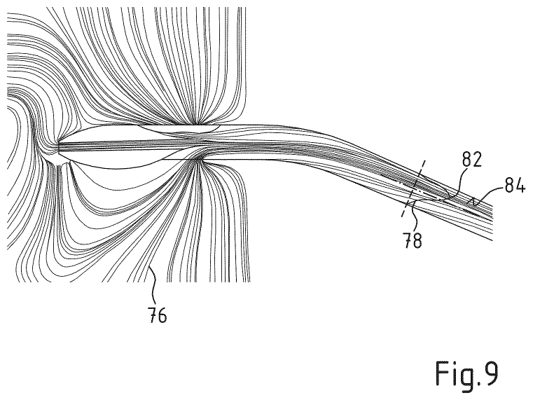

In a corresponding cardiac support system without the guide nozzle 37, in contrast to the blood drawn into the delivery channel 36, the flow lines 76 shown in FIG. 9 form with a flow profile 78 over the cross-section of the delivery channel, which has a maximum 82 near the channel wall 84.

The above-described arrangement and geometry of the guide nozzle 37 thus enables the gentle delivery of blood from a heart chamber by means of the cardiac assist system, because shear forces in the delivery channel 36, which are the cause of hemolysis, are minimized.

In one summary aspect, the cardiac assist system 10 has a pumping device 12 for pumping blood into a blood vessel through a catheter and comprising a pump housing 14 and a hollow body 22 connected to the pump housing 14, the hollow body 22 having a distal end portion 26 and an inlet portion 28 formed between the distal end portion 26 and the pump housing 14 with at least one inlet opening 30, 32, 34, a flexible hose portion 24 with a delivery channel 36 extending to the pump housing 14. The at least one inlet port 30, 32, 34 communicates with the delivery passage 36 through a guide nozzle 37 connected to an end of the hose portion 24 facing the distal end portion 26. The guide nozzle 37 has a constriction 42 formed between an inlet port 38 and an outlet port 40.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A cardiac support system comprising:
a housing comprising a pump configured to be introduced into a blood vessel through a catheter to pump blood from the left ventricle into the aorta, a hollow body connected to the housing and comprising a flexible hose portion with a delivery channel extending therethrough to the pump, a distal end portion connected to the hollow body, and an inlet portion located between the distal end portion and the hollow body, the inlet portion having at least one inlet opening configured to receive blood therethrough; and
a guide nozzle extending from a nozzle inlet opening, that is in fluid communication with the at least one inlet opening of the inlet portion, to an outlet opening that is in fluid communication with the delivery channel of the flexible hose portion, the guide nozzle connected to a distal end of the hose portion and facing the distal end portion, wherein the guide nozzle protrudes radially-inwardly along a contour to form a minimum-width constriction located between the nozzle inlet opening and the nozzle outlet opening, and wherein the contour has a single, continuous concavity or convexity along an entire length from the nozzle inlet opening to the nozzle outlet opening.

2. The cardiac assist system of claim 1, wherein the guide nozzle has a curved distal opening edge surrounding the inlet opening.

3. The cardiac support system of claim 2, wherein the opening edge at least partially defines the at least one inlet opening.

4. The cardiac assist system of claim 1, wherein the guide nozzle is inserted into the hose section.

5. The cardiac assist system of claim 1, wherein the guide nozzle is formed as a one-piece, molded part.

6. The cardiac assist system of claim 1, wherein the guide nozzle is composed of a plurality of shell-shaped moldings.

7. The cardiac assist system of claim 1, wherein the guide nozzle defines a nozzle channel extending along a nozzle axis having cross-sectional areas as measured perpendicular to the nozzle axis, and wherein the cross-sectional areas decrease from the inlet opening to the constriction and increase from the constriction to the outlet opening.

8. The cardiac assist system of claim 7, wherein the cross-sectional areas are circular or elliptical.

9. The cardiac assist system of claim 7, wherein the guide nozzle has a flow guide contour, which is located in the plane of a longitudinal section running along the nozzle axis in a Cartesian coordinate system with a coordinate origin lying on the nozzle axis and an abscissa lying on the nozzle axis as a line drawn in a first and/or second quadrant of the Cartesian coordinate system and is formed as a convex line and as a concave line extending in a third and/or fourth quadrant of the Cartesian coordinate system.

10. The cardiac assist system of claim 9, wherein the flow guide contour has a rounded apex facing the distal end portion.

11. The cardiac assist system of claim 9, wherein the convex line and the concave line are each continuously differentiable.

12. The cardiac assist system of claim 7, further comprising a flow guide body connected to the distal end portion and projecting proximally into the inlet portion.

13. The cardiac assist system of claim 12, wherein the flow guide body has a guide contour which is rotationally symmetrical to the nozzle axis.

14. The cardiac assist system of claim 12, wherein the flow guide body has a flow guiding contour, which is located in the plane of a longitudinal section extending along the nozzle axis in a Cartesian coordinate system with a coordinate origin lying on the nozzle axis and an abscissa lying on the nozzle axis as a line drawn in a first and/or second quadrant of the Cartesian coordinate system and is formed as a convex line and as a concave line extending in a third and/or fourth quadrant of the Cartesian coordinate system.

15. The cardiac assist system of claim 1, wherein a first axial distance AE from the inlet opening to the constriction is greater than a second axial distance AA from the constriction to the outlet opening.

16. The cardiac assist system of claim 1, wherein the guide nozzle comprises a stepped-down outer width configured to attach to an inner surface of the hollow body.

17. A cardiac support system comprising:
a pump configured to be introduced into a blood vessel through a catheter to pump blood from the left ventricle into the aorta;
a body having a proximal end fluidly connected with the pump and extending longitudinally to a distal end to at least partially define a flow channel; and
a guide nozzle located at the distal end of the body and in fluid communication with the body to at least further partially define the flow channel, the guide nozzle protruding radially-inwardly into the flow channel with a curvature having a single concavity or convexity along an entire length from an inlet to an outlet of the guide nozzle.

18. The cardiac assist system of claim 17, wherein a first axial distance AE, measured from the inlet opening to a minimum-width constriction of the flow channel within the guide nozzle, is greater than a second axial distance AA, measured from the minimum-width constriction to the outlet opening.

19. The cardiac assist system of claim 17, wherein the guide nozzle comprises a stepped-down outer width configured to attach to an inner surface of the body.

20. The cardiac assist system of claim 17, wherein the guide nozzle has a curved distal opening edge.
